(19) United States
(12) Patent Application Publication
(10) Pub. No.: US 2022/0268691 A1
(43) Pub. Date: Aug. 25, 2022

(54) MICROSCOPE ADAPTOR AND SAMPLE MOUNT FOR MAGNETICALLY ACTUATING SAMPLE

(71) Applicant: University of Southern California, Los Angeles, CA (US)

(72) Inventors: Andrea M. ARMANI, Los Angeles, CA (US); Kylie TRETTNER, Los Angeles, CA (US)

(21) Appl. No.: 17/677,617

(22) Filed: Feb. 22, 2022

Related U.S. Application Data
(60) Provisional application No. 63/151,897, filed on Feb. 22, 2021.

(51) Int. Cl. G01N 21/17; G01N 33/50

(52) U.S. Cl. CPC G01N 21/1717; G01N 33/5005; G01N 2021/1727

(57) ABSTRACT

Many samples (inorganic and organic) have magnetic properties. This adaptor which is comprised of an electromagnet and a sample holder (slide) can be directly mounted on a standard microscope (upright or inverted). The magnetic field is uniform across the sample and can be modified (due to the electromagnet design). The mount allows changing the field while simultaneously imaging the sample. Notably, the universality of the adaptor design will allow it to enable a wide range of investigations, impacting numerous fields.

### CROSS REFERENCE TO RELATED APPLICATION(S)

This disclosure claims priority to U.S. Provisional Patent Application 63/151,897, filed Feb. 22, 2021, which is hereby incorporated by reference in its entirety herein.

### GOVERNMENT SUPPORT

This invention was made with government support under institute contract/grant number N00014-17-1-2270awarded by the U.S. Office of Naval Research. The government has certain rights in the invention.

### FIELD OF THE INVENTION

Provided herein, in certain aspects, are systems and methods of designing, making, and using a microscope adaptor and sample mount system for magnetically actuating a sample.

### BACKGROUND

Optical light based microscopes are known. Various fixtures and/or sample holder have been developed to enhance the attachment of a sample to a microscope for better viewing. However, various issues with such fixtures and/or sample holders remain.

### SUMMARY

According to some embodiments, there is provided a system configured to facilitate magnetically actuating a sample while imaging the sample with a microscope. The system comprises an adaptor body configured to removably couple with a microscope stage and/or other components. The adaptor body comprises an orifice configured to pass light from a light source of the microscope, through the sample, to one or more imaging devices of the microscope. The system comprises a sample holder formed by the adaptor body and configured to hold the sample such that the sample is positioned to receive and pass the light from the light source to the one or more imaging devices. The system comprises an electromagnet integrated into one or more surfaces of the adaptor body, and/or coupled to the adaptor body in other ways. The electromagnet is configured to generate a magnetic field that interacts with the sample. The magnetic field is configured to be changed to actuate, and/or the magnetic field is modulated to change, the sample while the sample is being imaged.

In some embodiments, the electromagnet is configured to generate a uniform magnetic field across the sample. In other embodiments, the magnetic field may be a time-varying magnetic field or magnetic field gradient applied to the sample. In some embodiments, the electromagnet comprises a Helmholtz coil system. In some embodiments, the sample comprises a microscope slide (e.g., a glass slide or a plastic slide), a hydrogel, magnetic particles, and/or other components.

The system is designed to precisely control mechanical properties of a magnetic cell culture matrix and simultaneously measure its impact on cellular and organoid biochemistry and morphology to enable experiments across a wide range of biological fields. There are many applications for such a system. Such a system may be used for microscope imaging of almost any magnetically actuatable substance. For example, such substances may include hydrogels, magnetic semiconductor materials, and/or other materials.

As one possible example application, the present microscope adaptor and sample mount system may be used with a magnetically tunable hydrogel with actuatable mechanical properties (e.g. stiffness, porosity, tortuosity). Among other possibilities, these systems and methods may be used to investigate how physical changes in the system modify the vesicles (exosomes) that the tumor cells release (FIG. 1). Specifically, FIG. 1 shows a schematic of proposed objectives including establishment of hydrogel (i.e., magnetogel, also referred to as an MAGE hydrogel) to host Pancreatic ductal adenocarcinoma/PDAC-derived organoid, tuning physical properties through a magnetic field, and analysis of secreted exosomes. The dynamically tunable hydrogel matrix may improve experimental precision and reproducibility by removing sample to sample variation which is a critical confound in current methods. While the present example application is focused on pancreatic cancer, this technology will have broad applications because while many cancers (for example) exhibit extracellular matrix (ECM) stiffness, no one has developed an integrated system that overcomes both the inefficiencies of current ECM hydrogels and the limitations of exosome-based detection methods.

PDAC has a 5-year survival rate of 10% and is the third leading cause of cancer death in the United States. This poor survival rate is directly tied to the low effectiveness of current treatment options.

Because early stage PDAC is hard to detect due to its asymptomatic nature, most patients are already at the late stages of the disease when they are diagnosed. This results in less than 20% of patients being viable candidates for surgical resection. For the majority of patients, chemotherapy is the only treatment option. As such, response to therapeutics in this patient population is critical. However, PDAC progression is associated with increased fibrosis in the tumor microenvironment, such as an accumulation of extracellular matrix (ECM) proteins, resulting in mechanically stiff tumors. This phenomenon restricts drug delivery to affected areas and promotes resistance to cytotoxic therapies. Beyond the biophysical barriers present in the tumor microenvironment, altered intercellular communication contributes to increased chemoresistance. As such, this chemoresistance obstacle combines biochemical and biophysical features. Therefore, there is a critical need for more effective therapeutic strategies for PDAC.

Recent studies highlight the importance of exosomes in intercellular communication within the tumor microenvironment. Exosomes are extracellular vesicles ranging from 40-16 0 nm in size and contain microRNA, mRNA, DNA, and protein and are actively taken up by cells through receptor-mediated endocytosis. Many tetraspanins, including CD63, CD9, and CD81, are found on the surface of actively secreted exosomes but not extracellular vesicles like apoptotic bodies that are passively released following cell death. Moreover, exosome size, tetraspanin ratios, and pro-tumorigenic potential are known to change in response to changes in the microenvironment. Oncogenic exosome release is affected by fibroblasts, cells in the tumor microenvironment that synthesize and deposit ECM proteins leading to a stiff environment in PDAC. This indicates that PDAC is governed by both biochemical and biophysical cues. PDAC cells react to the presence or absence of the ECM. However, no biomimetic models currently have the critical ability to dynamically alter the ECM to quantify the biophysical changes necessary to induce biochemical responses.

Specifically, to study the role of the microstructure on pancreatic cancer cells or organoids, it is important to dynamically modulate or tune the structure in a non-invasive manner. While static systems have been developed, a dynamically tunable microstructure has not been demonstrated. The present microscope adaptor and sample mount system for magnetically actuating a sample provides this and/or other functionality.

Additionally, continuing with this example application, the present microscope adaptor and sample mount system for magnetically actuating a sample facilitates detecting and profiling exosomes from pancreatic cancer organoid cultures. A challenge was related to the large amount of media supernatant required. Many current technologies require a large amount of starting material (upwards of 50 ml) which is virtually impossible to gather from organoid cultures. Therefore, in spite of evidence showing the critical role of exosomes in PDAC, the effect dynamic ECM changes on the exosome secretion of pancreatic cancer organoids has not been elucidated. Therefore, a multi-parametric analysis incorporating the real-time response of PDAC to modifications in chemical and mechanical properties of the microenvironment is needed. Accomplishing this required the development of new investigative tools, such as the present microscope adaptor and sample mount system, which is capable of dynamically altering the environment around PDAC model systems.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows a schematic of establishment of a hydrogel to host PDAC-derived organoids, tuning physical properties through a magnetic field, and analysis of secreted exosomes, per embodiments herein.

FIG. 2A shows a PDAC-derived organoid formation in hydrogel culture, in accordance with an embodiment herein. FIG. 2B shows optical images of organoids grown in M-gel matrix before (top) and after (bottom) GEMM treatment.

FIG. 3 is a pictoral display of an embodiment of the system described herein.

FIG. 4 is a schematic showing an overview of iterative material development and an optimization approach in accordance with embodiments herein.

FIG. 5 shows a schematic example of an embodiment of an electromagnet system integrated sample stage for imaging, incorporated with a microscope and its stage.

FIG. 6 shows a detailed view of an adaptor and the electromagnet system mounted in a microscope, in accordance with an embodiment.

FIGS. 7, 8, 9, and 10 illustrate an isometric view, a side (right) view, a top view, and a front view, respectively, of an adaptor of the electromagnet system in accordance with an embodiment herein.

FIG. 11 illustrates an isometric view of the adaptor shown in FIGS. 7-10, in accordance with an embodiment.

FIG. 12 illustrates the adaptor of FIGS. 7-10 and another embodiment of an adaptor, both including coils or wires.

FIG. 13 illustrates an isometric view of an exemplary embodiment of another adaptor of the electromagnet system in accordance with an embodiment herein.

FIG. 14 illustrates a side view of the adaptor of FIG. 13, without coils or wire therein.

FIG. 15 illustrates an isometric view of the adaptor of the FIG. 13.

FIG. 16 is a schematic of an exemplary design for moving a slide with a sample within an adaptor, in accordance with an embodiment.

FIG. 17 is a schematic of an exemplary design of cooling means provided within an adaptor, in accordance with an embodiment.

FIG. 18A illustrates an example embodiment of an electromagnet system with integrated sample stage/adaptor, in accordance with another embodiment, for imaging with emphasis on how the electromagnet system may be incorporated with a microscope stage. FIG. 18B illustrates an alternative example embodiment of an adaptor for imaging.

FIG. 19 illustrates a schematic illustration of a proposed workflow described herein. A supernatant from gel cultures may be collected and analyzed through ExoView™, for example. Exosome purification may be performed and added to a cell culture without a pre-existing exosome to analyze the exosome function in terms of proliferation and chemo-resistance.

FIG. 20 illustrates an example embodiment of a computer system that may be used in conjunction with any of the operations described herein.

### DETAILED DESCRIPTION

This disclosure will now be described more fully hereinafter with reference to the accompanying drawings in which exemplary embodiments of the disclosure are shown. However, the disclosure may be embodied in many different forms and should not be construed as limited to the representative embodiments set forth herein. Reference to various embodiments does not limit the scope of the claims attached hereto. Like reference numbers refer to like elements throughout the various drawings and views. Additionally, any examples set forth in this specification are not intended to be limiting and merely set forth some of the many possible embodiments for the appended claims.

To mitigate the problems described herein, the inventors had to both invent solutions and, in some cases just as importantly, recognize problems overlooked (or not yet foreseen) by others. Indeed, the inventors wish to emphasize the difficulty of recognizing those problems that are nascent and will become much more apparent in the future should trends in industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

The present disclosure describes a system configured to facilitate magnetically actuating a sample, while imaging the sample with a microscope. The system comprises an adaptor (or adapter) body configured to removably couple with a microscope stage and/or other components. The adaptor body comprises an orifice configured to pass light from a light source of the microscope, through the sample, to one or more imaging devices of the microscope. The system comprises a sample holder formed by the adaptor body and configured to hold the sample such that the sample is positioned to receive and pass the light from the light source to the one or more imaging devices. The system comprises an electromagnet integrated into one or more surfaces of the adaptor body, and/or coupled to the adaptor body in other ways. The electromagnet is configured to generate a magnetic field that interacts with the sample. The magnetic field is configured to be changed and/or modulated to actuate and/or change the sample while the sample is being imaged.

In some embodiments, for example, a Helmholtz coil system may be adapted to fit on a microscope stage to allow for magnetic actuation while imaging. The system may be optimized around ensuring a magnetic field across an entire sample, while leaving space for optical (fluorescent) imaging. The design accommodates a microscope slide and ensures a transparent surface for imaging applications. The design also supports horizontal and vertical application of a magnetic field and can be configured in both directions.

As one possible example application (there are many other different possible applications), the present microscope adaptor and sample mount system may be used with a magnetically actuatable hydrogel with tunable mechanical properties (e.g. stiffness, porosity, tortuosity) to form a dynamically tunable environment based on a hydrogel matrix that will meet the critical need described above and/or other needs. Hydrogels are three-dimensional (3D) crosslinked polymers imbued with solvent. This general material architecture can be used in in vitro ECM models. To create a dynamically tunable system, a novel magnetic hydrogel ECM matrix was created to use as a platform for detecting how dynamic ECM changes affect PDAC organoid tumorigenesis via exosome characterization. Dynamically altering the supporting microstructure of a PDAC organoid through the application of a tunable magnetic field addresses the need for mechanical complexity in ECM models. The present systems and methods enable the quantitative analysis of the ECM's contribution to pancreatic cancer development, improve reproducibility in biological investigations, transform the approach to PDAC therapeutic development, and/or have other applications. The present systems and methods comprise a magnetically responsive hydrogel ECM model, dynamic tunability of the hydrogel matrix, quantification of the changes in hypersecreted exosomes derived from PDAC organoids cultured in the hydrogel matrix, and/or other functionality.

3D Dynamic Cell Cultures

Continuing with this example application, traditional two dimensional (2D), monolayer culture of cancer cells on plastic dishes was the mainstay methodology in basic and translational research for decades. Despite some therapeutic discoveries made by performing drug screening on cells grown in 2D, these simple models, which lack the complexity and cellular heterogeneity found in actual tumors, often fail to accurately model critical responses from the tumor microenvironment that ultimately leads to chemoresistance in patients. Hence, the development of models that better recapitulate the key features of human cancers is critical to advance the efforts to discover effective therapeutic regimens. To accomplish this task, the present system is 3D. This required creating 3D cell culture matrices as well as 3D cell structures.

Matrigel™ is one example in a class of hydrogel, or crosslinked polymers imbued with solvent, that are often used as ECM mimics for complex cellular experiments. As a commercially available system, Matrigel has clear advantages. However, it is produced from a biological system, leading to substantial batch to batch variation which directly impacts the reproducibility of the biological findings. To address these issues, designer matrices based on a wide range of user-defined formulations have emerged, and researchers have successfully cultured organoids. One popular material system is a simplified formulation with only type IV collagen and/or hyaluronic acid. However, these systems still rely on mostly biological components. Additionally, while the microarchitecture can be changed discretely for each batch, dynamic or in situ microarchitecture control is not possible. A system that utilizes forces exogenous from what is common in cellular biology has a distinct advantage of more specific control over the system.

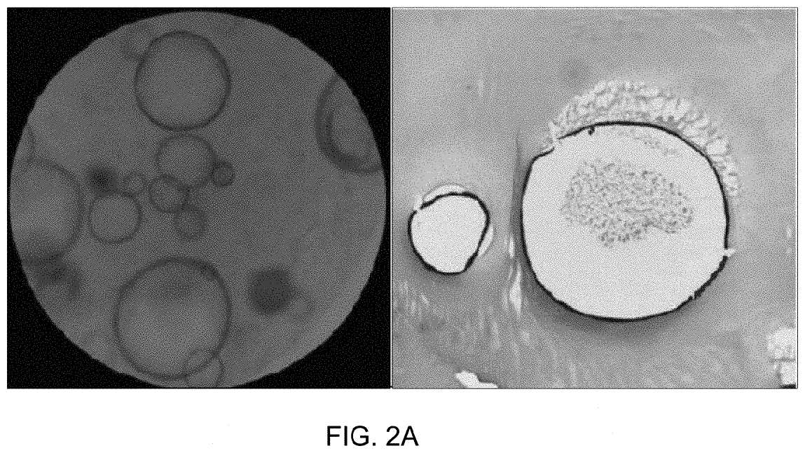

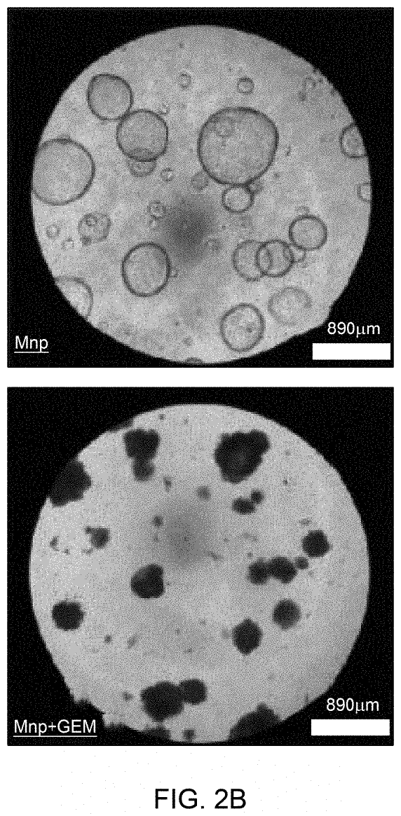

The initial 3D cell cultures were simply cellular clusters or spheroids. As the field advanced, researchers developed organoids. These 3D systems maintained some function of the primary cell-type. More recently, the patient-derived organoid (PDO) cultures have emerged as a powerful biomimetic model in cancer research. In pancreatic cancer research, PDOs are derived from tumors of PDAC patients or genetically engineered mouse models (GEMM) of PDAC. FIG. 2A shows an example phase-contrast image of a PDAC-derived organoid growing in a hydrogel system (left image). FIG. 2A also shows (right image) an image after paraffin embedding and 5 μm section preparation. H&E staining confirmed physiologically relevant gland morphology of cultured organoid. FIG. 2B shows optical images of organoids grown in M-gel matrix before (top) and after (bottom) GEMM treatment. Traditionally grown in Matrigel™ as three dimensional (3D) multicellular structures, PDOs have been shown to retain the molecular signature of the pancreatic tumor tissue that they were derived from, and prior work has relied on the traditional Matrigel™ environment. As a result, the majority of the work to date has focused on assessing how PDOs react to the presence or absence of the ECM. This limits the possible investigations because the PDAC matrix is observed as a gradually stiffened environment, and few of the existing hydrogel designs provides a matrix that allows dynamically tuned microstructure changes in organoid cultures.

Magnetogel Toxicity Study

To demonstrate the biocompatibility of the magnetogel (M-gel) system, two Matrigel constructs with different nanoparticle concentrations (1 mg/mL and 0.5 mg/mL) were made. Pancreatic spheroids were grown on a 96-well culture plate. All measurements were performed in sextuplet (6×). The viability from these wells was compared against two controls: Matrigel/cells only, and M-gel/cells treated with 10 μL of gemcitabine (GEM), a common chemotherapeutic agent (as shown in FIG. 2B). The resulting data shows a stark difference between the viability of cells killed by GEM control and the non-significant (ns) difference in viability between the standard Matrigel control and the M-gels, thus suggesting the biocompatibility of the matrices.

Hydrogels provide the added benefit of introducing specific functionality by engineering the polymers, surface, and environmental responsiveness of the constituent material. Externally controlled, or stimuli responsive, hydrogel systems provide an opportunity to address the biophysics of PDAC tumor fibrosis. Among the possible physical forces, electrical, mechanical, and magnetic rise to the top, due to their intrinsic compatibility with imaging systems. However, electrical could cause confounds with endogenous bioelectric fields. While easy to implement, mechanical compression methods across a viscoelastic material, such as a hydrogel, result in a nonlinear distribution of force, which is not ideal. Therefore, magnetic field actuation has the clear advantages of reducing confounds, improving uniformity across the sample (i.e., when a uniform magnetic field is applied), and low cytotoxicity.

To synthesize a magnetically controlled hydrogel, magnetic nanoparticles can be integrated into the hydrogel matrix, either simple intercalation or directly conjugated to the polymers. The mechanistic changes to the matrix microstructure upon magnetic influence are multifaceted as rearrangements of the matrix may have microscale effects on the porosity, tortuosity, and resultant stiffness. Thus, either synthesis approach addresses the spatiotemporal limitation of current, static hydrogel systems.

Exosome Analysis

Exosomes are extracellular vesicles that play an important role in intercellular communication in the tumor microenvironment. Increased exosome release (hypersecretion) is caused by cells in response to a variety of cell intrinsic and cell extrinsic stress cues. These exosomes transport cancer-promoting factors (e.g. miRNA, RNA, DNA, circular RNAs, protein) from cancer cells to recipient cells. However, the quantity of exosomes is not the only important factor in exosome signaling. Exosomes are enriched with surface tetraspanins proteins like CD9, CD63 and CD81. These tetraspanins serve as exosome markers, facilitate vesicle biogenesis and more importantly, participate in cargo selection. Hypersecretion of exosomes can produce different subpopulations of exosomes based on different stimuli. Exosome size, tetraspanin ratios, and pro-tumorigenic potential are known to change in response to changes in the microenvironment. Thus, when studying exosome production as a biological readout, the key relevant data are size distribution, frequency, tetraspanin ratio, and content.

While a high sensitivity immunoassay could be used to obtain information about exosome presence, all information regarding exosome size and frequency would be lost. Additionally, by relying solely on exosome concentration as a readout, incorrect conclusions could easily be drawn. For example, are multiple small exosomes or a single large exosome being produced, as either scenario could generate the same quantity of a protein. Moreover, these two scenarios could be indicative of two different biochemical or biophysical pathways and could lead to different therapeutic strategies being proposed. Therefore, novel technologies capable of generating comprehensive data sets from isolated exosomes are needed.

To overcome this barrier, the present systems and methods leverage the Exoview System. This instrument can quantify exosome size, number and tetraspanin profile using as little as 35 μl of sample. Therefore, the present systems and methods are able to quantitatively and comprehensively correlate exosome production from PDAC organoids to hydrogel stiffness, something that would not be possible using traditional exosome isolation and analysis methods. Through the use of the present magnetically tunable hydrogel matrix (described herein) in combination with the Exoview technology, the present (dynamic) system makes it possible to analyze the production of exosomes in response to changes in microarchitecture.

Commercialization Potential

The present systems and methods comprise experimental tools configured for investigating the relationship between physical changes in cancer and exosome hypersecretion. The present systems and methods comprise 1) the development of a transformative technology to dynamically and non-invasively modulate the hydrogel stiffness, 2) the use of comprehensive exosome analysis as a functional readout for studying the consequence of ECM stiffness on PDAC organoids, and/or other aspects. This integrated technology has applications across multiple cancer systems. Specifically, components of the present hydrogel system (e.g., the magnetically tunable material and the electromagnet-based microscope adaptor) have never been developed before, and facilitate opportunities with impact across multiple market sectors. Further, as will be evident by the exemplary embodiments below, the disclosed system is designed to dynamically modulate the mechanical stiffness in a 2D culture plate or a 3D culture network, and is also compatible with standard confocal fluorescence microscope stage and imaging objectives, enabling simultaneous real-time imaging and real-time manipulation of the microenvironment.

Patient Population

Approximately 47,050 people in the US died of pancreatic cancer in 2020 according to data provided by the National Cancer Institute's Surveillance, Epidemiology, and End Results program. The disease currently has a five-year survival rate of only 10%, the worst of any major cancer. Late detection which prevents surgical intervention is a major cause of this poor prognosis. Even in the patient population eligible for surgery, the standard-of-care post-resection is either gemcitabine (GEM) in combination with Nab-paclitaxel (NPT) or folfirinox (FOL). Unfortunately, response to GEM-based treatments is observed in only 37% of patients. While FOL exhibited better patient response than GEM alone, it also resulted in severely increased toxicity, so it is not suitable for all patients. Moreover, patients on FOL who inevitably relapse are then put on GEM-based combination treatments. Therefore, there is a critical need for more effective therapeutic strategies for PDAC. The feasibility of the present systems and methods has been demonstrated in commercially available pancreatic cancer cells and organoids.

Magnetically Functionalized Hydrogel ECM Model

As noted, the disclosed system includes two complementary components: magnetogel and an electromagnet imaging mount (or adaptor). While either concept could stand alone, when used in concert, they transform the role of the microscope stage in imaging. Integration of magnetic nanoparticles to a hydrogel base — to form a magnetically-actuatable hydrogel (magnetogel)—provides a biocompatible product that is transparent and which may be actuated (and/or changed) by the integrated electromagnet of the system to enable simultaneous, real-time actuation while performing in situ imaging. Current systems for 3D cell culture and organoid growth rely on natural polymers that have non-negligible variability between lots. This system focuses on allowing for a greater degree of tunable functionalization and the opportunity to customize the matrix.

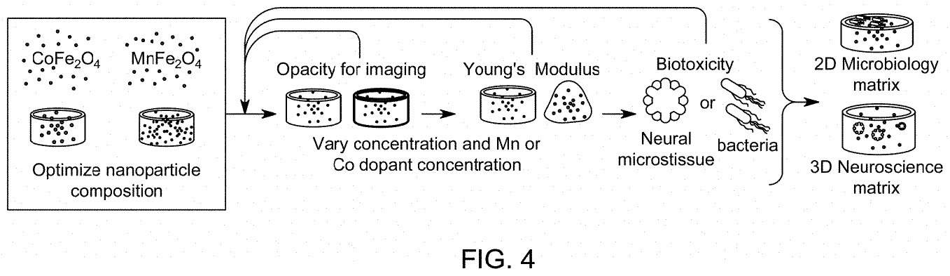

Manganese-doped iron oxide (MnFe2O4) nanoparticles (NPs) or cobalt-doped iron oxide (CoFe2O4) NPs may be utilized in the hydrogel/magnetogel, in accordance with an embodiment. While previous work has focused on iron oxide, due to the simplicity of the synthesis, MnFe2O4 or CoFe2O4 are selected because of their higher magnetic susceptibility. This will allow lower concentrations to be used in the hydrogel, improving imaging quality. By using Co- or Mn-doped iron oxide nanoparticles with high magnetic susceptibility, biocompatible and optically transparent hydrogels capable of tissue growth may be synthesized. Synthesis may be performed using standard air-free techniques according to published protocols, with varying dopant concentration. Such is schematically illustrated in FIG. 4, which shows an overview of iterative material development and an optimization approach for modifying biocompatible magnetogels to be compatible with microtissues. As an example, manganese and iron precursors may be reacted in a 1:2 ratio with an organic ligand before exchanging the hydrophobic groups for hydrophilic moieties to allow for monodispersion of the particles within the hydrogel matrix. A similar route may be used for the Co-doped nanoparticles. To synthesize a magnetically controlled matrix, surface functionalized MnFe₂O₄ or CoFe₂O₄ nanoparticles will be mixed into the hydrogel solution before curing, resulting in nanoparticles being diffused throughout the hydrogel matrix. Since some tissues require unique material host systems, collagen-I or agarose may be utilized as a host matrix. After synthesis, the magnetic response may be characterized using a magnetometer, and the size distribution of NPs will be measured using electron microscopy and dynamic light scattering. For example, the magnetometer may be used to analyze field strength, field uniformity, and switching speed. In an embodiment, target quantitative milestones may be the following: field strength—maximum of 0.25 T, field uniformity—less than 0.01 T variation across a 4 cm sample, and switching speed—maximum of 1 Hz. To analyze the switching speed (as well as several system response parameters), an output signal from a function generator may be connected to an oscilloscope, and the signal from the magnetometer may be connected to a computer (via USB). Both systems can record in excess of kHz switching speeds which greatly exceeds the values needed for these measurements. Using fluorescent nanoparticles as tracer particles, validates the ability to perform confocal imaging (fluorescent) while dynamically tuning (or moving) the magnetogel matrix.

Due to the nanoparticles, the hydrogels are magnetically responsive, changing their mechanical properties in response to magnetic field strength. To support research across the bio community, different hydrogel formulations may be designed and optimized. Implementing such a controllable magnetogel system enables measurement of the response of cells to both spatial and dynamic changes of gel stiffness. Migration of cells living on gel substrates is regulated by the mechanical properties of the gel. The effective rate at which cells disperse through the gel is impacted by the mechanical properties of the gel. Accordingly, the rate and strategy for cell migration changes, as the gel stiffness and porosity is dynamically tuned, may be observed in the resulting magnetogel via the disclosed system and adaptor.

Intercalating the magnetic NPs into a hydrogel supports the particles in a manner than allows them to freely polarize and reduces the diffusivity of the gel system when influenced by a magnetic field. As this method does not involve further polymer functionalization, systems and methods may use this approach.

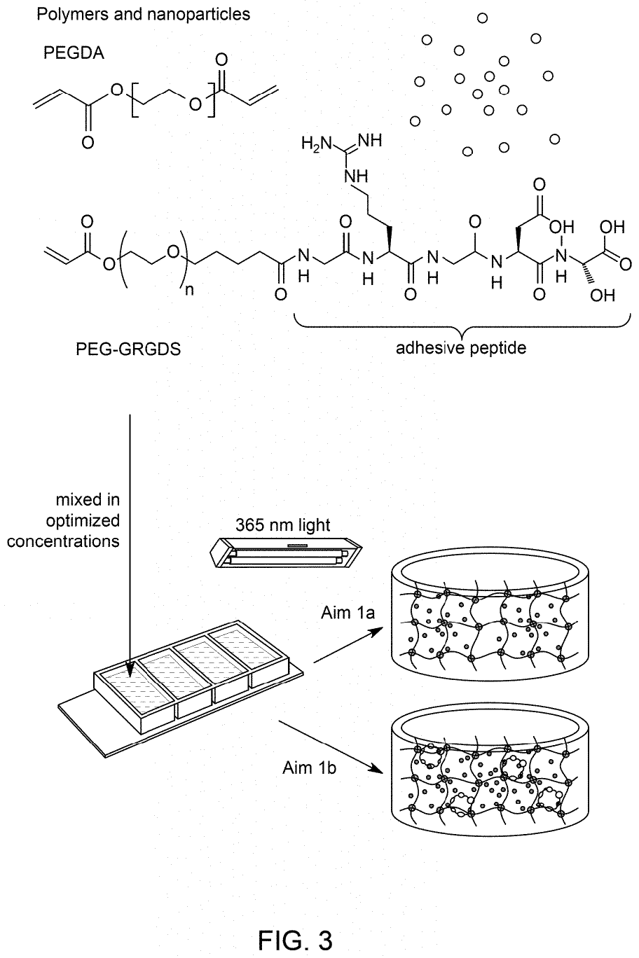

In order to use the magnetogel as a dynamic matrix, there are several variables (e.g., biologically relevant features, imaging characteristics, and magnetic response) that must be fine-tuned. Accordingly, the hydrogel matrix should be optimized for a given magnetic response for organoid growth. Continuing with this example application, there are a handful of variables that may be fine-tuned to support the growth of organoids. These include the overall and specific polymer concentrations, the appropriate concentration of nanoparticles and, if applicable, the amount of time the gel(s) are exposed to UV radiation, as displayed in the conceptualized hydrogel in FIG. 3, for example. In the development process, common statistical design methods may be employed to ensure the gel is optimized in a robust manner. One method commonly applied to industrial processes, the Taguchi method, involves design of experiments to optimize the factors of interest and then identify possibly sources of noise. Combining this proven technique with response surface methodology analysis may allow for the development of a robust, and reproducible matrix specifically designed to support organoid culture. Further measurements may be used in order to optimize the hydrogel. For example: the magnetogel's compatibility with fluorescent confocal imaging may be determined by embedding fluorescent nanoparticles of varying diameter (5 μm to 10 nm) with a resolution goal of 200 nm, for studying features of microtissues. The magnetic tunability of a hydrogel's stiffness may be characterized by varying the magnetic field in discrete increments and analyzing the Young's modulus change. The magnetic field will be applied using the electromagnet adaptor (described later below). A series of hydrogels with different nanoparticle concentrations may be formulated, establishing different “center” stiffness values.

These methods may be configured to ensure the robustness of the system and allow establishment of clear quality control (QC) measures to better track the reproducibility of organoid growth within the matrix. These variables may be evaluated according to the best evaluated growth of the organoids through fluorescent staining and comparison of hydrogel-derived organoids. Once optimized for best growth, the material properties of the hydrogel may be characterized and used to determine the modulus through load-frame testing, swelling ratio, and porosity and particle distribution (of dried matrix) via electron microscopy.

Confirm Dynamic Magnetic Tunability of the Hydrogel Matrix

Once the hydrogel is optimized for organoid growth and in vitro experiments, the dynamic behavior of the system may be confirmed. The application of a magnetic field, and the subsequent alignment of the nanoparticles with the applied field, may result in local microstructural changes to the matrix. Removing the magnetic field may provide an opposite effect. By controlling the magnitude of the magnetic field, through changes in applied current through the coils, the magnitude and directionality of the force applied to the environment surrounding the PDAC organoids is controlled.

Electromagnet System for Magnetic Field Application

A system configured to facilitate magnetically actuating a sample, while imaging the sample with a microscope, is provided. Notably, instead of the stage simply being a platform for a sample, the disclosed stage/adaptor will be able to dynamically expose a sample to an oscillating (or static) magnetic field. The static and dynamic (oscillating) system performance for the electromagnet may be characterized using the magnetometer in x-y-z axis and compared with FDTD modeling and particle tracking measurements. The disclosed design is compatible with both upright and inverted fluorescence microscopes, providing additional degrees of freedom.

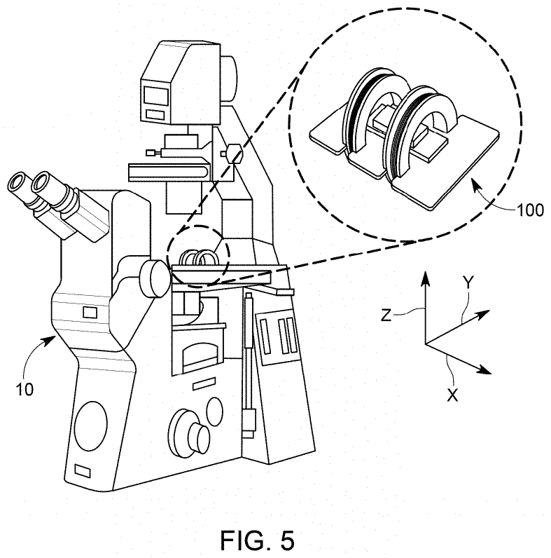

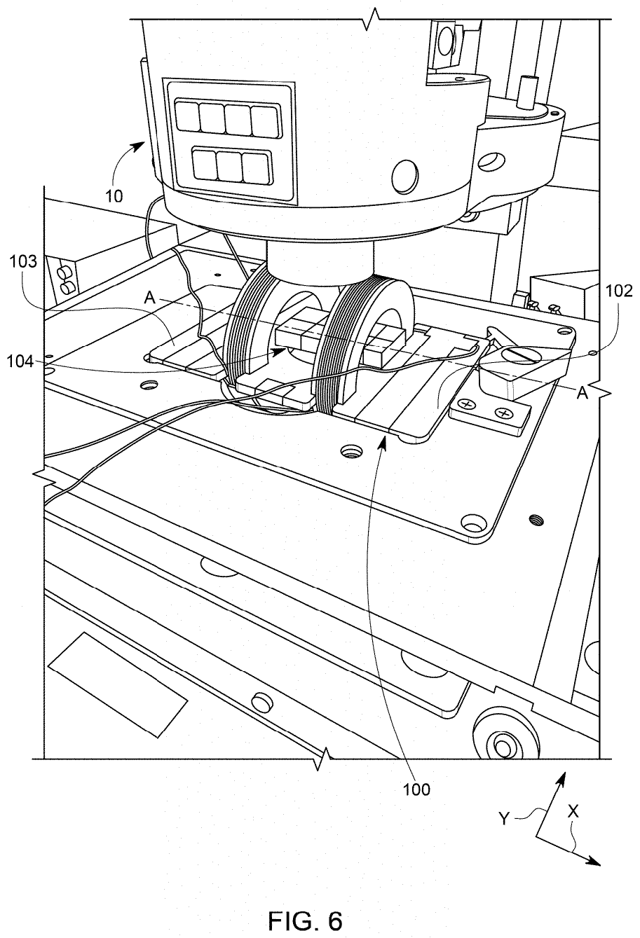

An example is illustrated in FIG. 5, showing a microscope 10 that has a stage with an integrated electromagnet, provided in the form of an electromagnet sample holder, also referred to here-throughout as an adaptor 100. FIG. 6 shows a detailed view of such an adaptor mounted in/on a microscope (in this exemplary case, on an Olympus confocal microscope). Generally, the microscope is designed with a light source (e.g., laser), mirrors, detector/imaging devices, objective lens, and pinhole(s), as understood by a person of skill in the art and thus not described in detail herein. Specifically, the system includes an adaptor with an adaptor body 102 configured to removably couple with a microscope stage and/or other components. The adaptor body 102 includes a base 103 which is configured to hold a conventional microscope slide 104 and to directly mount into any microscope stage, thereby creating a “universal stage mount” or “universal adaptor” for integration into any upright or inverted microscope(s) that is configured to simultaneously hold a biological sample cultured on a magnetogel and apply an oscillating magnetic field, for controlling the magnetically response matrix and imaging the same. As will be evident the described features detailed below, the adaptor is designed to ensure a magnetic field is applied (uniformly, time-varying, gradient) across an entire biological sample while leaving space for optical (fluorescent) imaging. The adaptor body may have many shapes provided any such shape functions as described herein. The adaptor body may be configured to removably couple with the microscope by a shape of the adaptor body (e.g., shaped slots, hooks, orifices, etc. formed in the body); via clips, clamps, hooks, screws, magnets, and/or other external components; and/or by other coupling mechanisms or mechanical devices. Dimensions of the adaptor and its body may be configured or determined based on the type or size of sample, and/or configured or determined based on the type and/or dimensions of the microscope (and the microscope stage that the adaptor is removably attached to). Some examples may be provided below but are not intended to be limiting in any way.

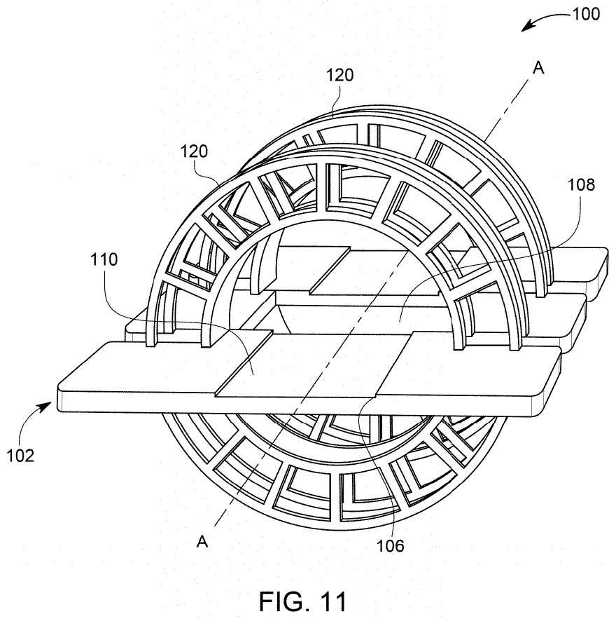

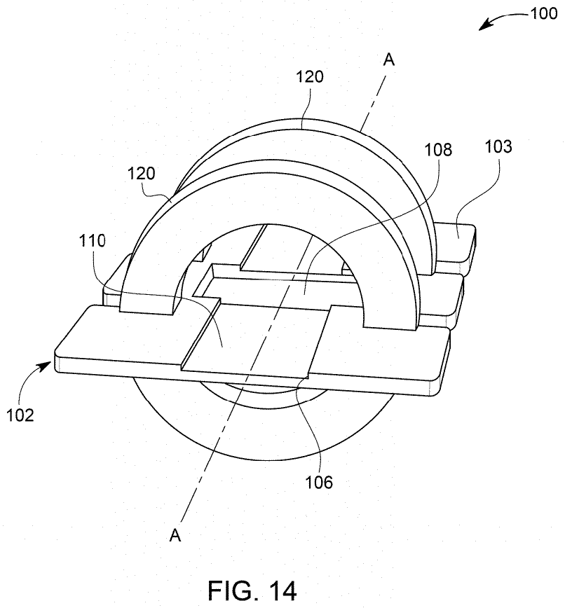

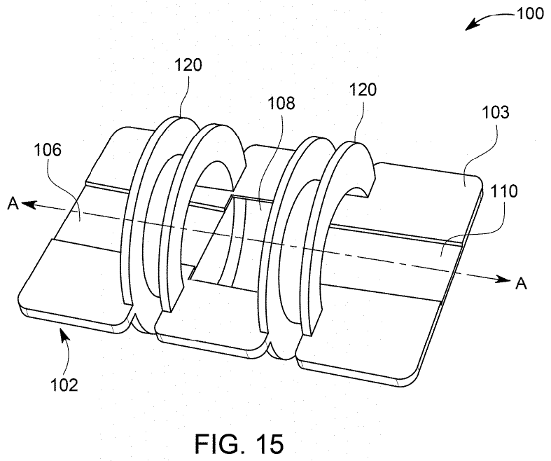

For example, the adaptor body may be configured to be removably attached to a microscope by one or more clamps. The adaptor body may be formed form metals, polymers, ceramics, and/or other materials. For example, the adaptor body may be formed from resin(s) (e.g., high temperature resins, ceramic resin), thermoplastic(s), steel, aluminum, titanium, an alloy metal, polycarbonate, Bakelite, and/or other materials. In some embodiments, the adaptor body may be formed from a combination of materials. The materials of the adaptor body may be configured such that the adaptor body is relatively light weight and easy for a user to removably couple with a microscope. The method of manufacturing the adaptor is not limited. In accordance with embodiments, the adaptor may be printed (e.g., 3D printing), molded, and/or casted, for example, either as an integral piece (i.e., a single formation) or as a piece that is made integral by connecting parts thereof together. Further exemplary features may be discussed below with reference to the non-limiting exemplary embodiments of an adaptor as shown in FIGS. 6-18. While features may be discussed with reference to a single embodiment or Figure, it should be noted that such features may be applied to other embodiments disclosed herein, even if not explicitly referenced or shown in a particular FIG.

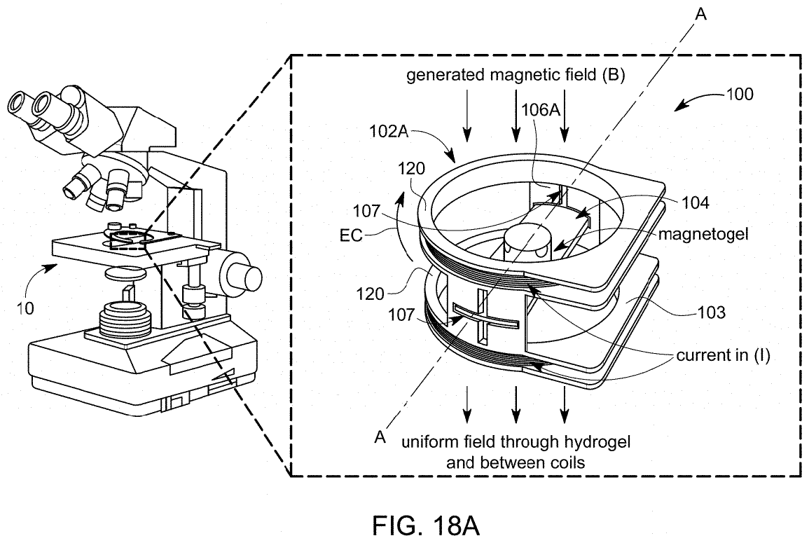

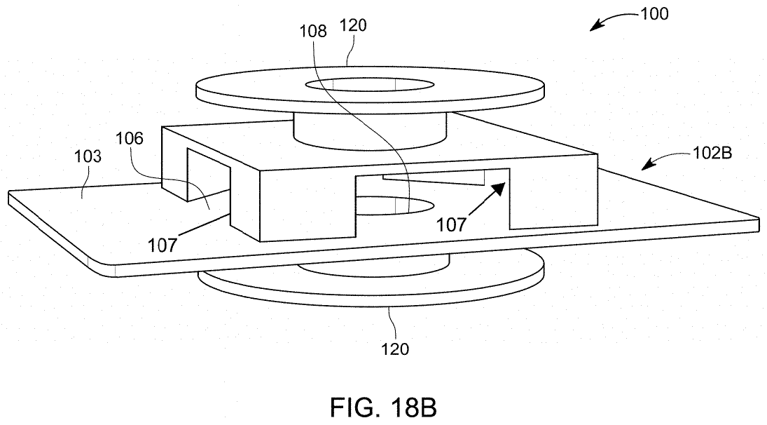

The base 103 of the adaptor body 102 may be configured to extend in a longitudinal (e.g., horizontal) direction along an axis (A-A), as represented in FIGS. 6, 7 and 9, in accordance with embodiments herein. Accordingly, in embodiments, the base 103 is configured for mounting in a generally horizontal configuration into a stage of a microscope, as shown in FIG. 6, for example. In an embodiment, such as shown in FIG. 18A or 18B, the base 103 may be configured for mounting in a horizonal direction such that the base 103 is on a side (e.g., right side as depicted in the FIG.), or rotated 90 degrees about axis A-A such that the base is positioned at a bottom (as depicted in the FIG.).

The system comprises a sample holder 106 formed by the adaptor body 102 and configured to hold the sample (i.e., slide 104) such that the sample is positioned to receive and pass light from a light source to the one or more imaging devices of the microscope, when the adaptor is mounted therein. The sample holder 106 may be configured to extend along a length i.e., in the longitudinal direction along axis A-A, of the base 103. In an embodiment, the sample holder 106 may include a recessed portion 110 (or slide inset) within the base 103 of the adaptor body 102, sized in accordance with conventional microscope slides, for receipt of a slide with a sample therein. As shown in FIG. 8, for example, this recessed portion 110 extends vertically downwardly into the base 103 to provide an offset such that the sample rests directly (or as close as possible to) a middle (r=0) when the slide is inserted therein. In an embodiment, a height HS and a width WS of the recessed portion 110 corresponds to a height and a width of a microscope slide. In one embodiment, the height H is approximately 1.0 mm to approximately 2.0 mm, and the width W is approximately 25.0 mm to approximately 30.0 mm. However, such dimensions of the recessed portions 110 are exemplary only and not intended to be limiting. Such a recessed portion 110 is optional, however. That is, the base 103 itself may be substantially flat and configured to form a surface that acts as the sample holder. FIG. 13 shows an example of a sample holder 106 in adaptor body 102 without such a portion. Additionally and/or alternatively, the sample holder 106 (or base 103) may be provided in the form of clips, snaps, or any other device that will physically hold the sample/slide to the base 103 of the adaptor, and such devices may be provided with or without the recessed portion 110.

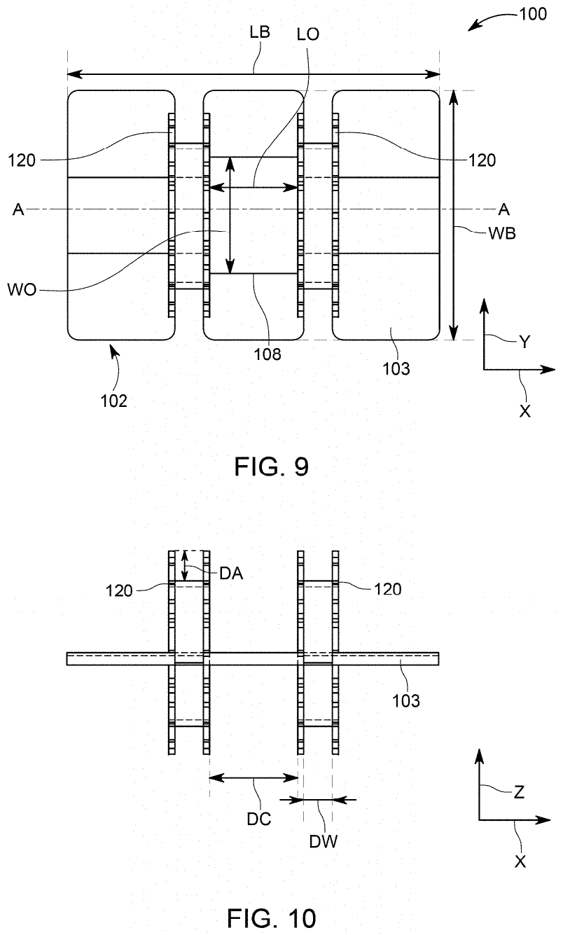

Dimensions of the base 103 may vary, and may, in accordance with embodiments, be based on the type of microscope utilized with the disclosed system. As depicted in FIGS. 8 and 9, the base has a height HB, a length LB, and a width WB. In an embodiment, the height HB of the base may be approximately 3.0 mm +/−0.5 mm to approximately 6.0 mm +/−0.5 mm. In an embodiment, the length LB of the base may be approximately 75.0 mm +/−1.0 mm to approximately 400.0 mm +/−10.0 mm. In another embodiment, the length LB may be approximately 125.0 mm +/−2.0 mm to approximately 300.0 mm +/−10.0 mm. In an embodiment, the width WB of the base may be approximately 25.0 mm +/−2.0 mm to approximately 300.0 mm +/−10.0 mm. In another embodiment, the width WB may be approximately 85.0 mm +/−5.0 mm to approximately 100.0 mm +/−5.0 mm. In an embodiment, the dimensions of the base 103 may be configured or determined based on the type or size of sample, and/or configured or determined based on the type and/or dimensions of the microscope (and the microscope stage that the adaptor is removably attached to).

In a particular non-limiting embodiment, the base may have a length and width of approximately 128 mm×86 mm (both +/−5.0 mm), and height of approximately 4.0 mm +/−0.5 mm. In another embodiment, the dimensions for the base may be expanded up to approximately 400 mm×300 mm (both +/−10.0 mm). In another embodiment, the dimensions for the base may be reduced to approximately 75 mm×25 mm (both +/−5.0 mm).

Moreover, the shape of base 103 is not intended to be limited. While a shape of the base 103 as shown in the Figures may be generally rectangular, the shape of the base may alternatively be square, circular, round (e.g., as shown in FIG. 18A), ovular, ellipsoidal, etc.

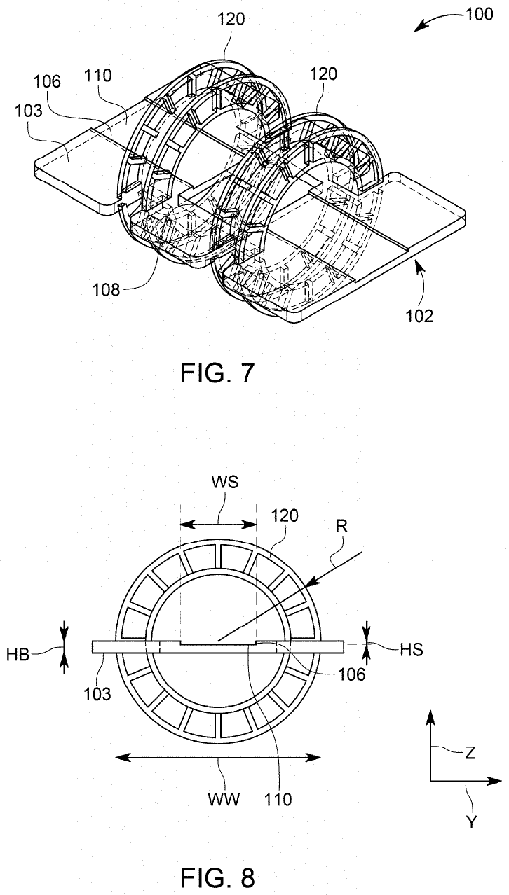

Also, as seen in FIGS. 7 and 9, the base 103 of the adaptor body 102 has an orifice 108 or imaging window therein which is configured to pass light from the light source of the microscope, through the sample (on slide 104), to the one or more imaging devices. The orifice 108 may be provided in a central area of the sample holder 106 and may be provided within the optional recessed portion 110, if provided. In an embodiment, the orifice 108 is rectangular in shape, such as shown in FIG. 9, having a length LO and a width WO. In an embodiment, the orifice 108 may have a length LO of approximately 30.0 mm and a width WO of approximately 40.0 mm. However, such dimensions are exemplary only and not intended to be limiting. The orifice 108 may have changes in size (larger, smaller) and/or shape (circular (such as shown in FIG. 13 and FIG. 18A-B), square, round, ovular, ellipsoidal, etc.). Again, the size of the orifice 108 may be configured or determined based on the type or size of sample, and/or configured or determined based on the type and/or dimensions of the microscope (and the microscope stage that the adaptor is removably attached to).

FIG. 18A shows an example of another embodiment of an adaptor with an adaptor body 102A configured to removably couple with a microscope stage and/or other components. The adaptor body 102A includes a base 103 which is configured to hold a conventional microscope slide 104 therein via sample holder 106A, such that the sample is positioned to receive and pass light from a light source to the one or more imaging devices of the microscope, when the adaptor is mounted therein. In an embodiment, the sample holder 106A may be formed by corresponding slots 107 provided in opposite sides of the adaptor body. In some embodiments, the slots 107 may be sized and/or shaped such that a microscope slide 104 may be slid into or out of the slots. When a slide (which supports a sample) is held by the slots 107, as shown in FIG. 18A, light from the microscope may pass through the slide and sample, and to the lenses of the microscope for imaging. The light may pass through the slide and sample while magnetic particles in the sample are being actuated (and/or changed) by the electromagnet (described below) of the system. Although not shown in FIG. 18A, it should be understood that the adapter body 102A includes an orifice or imaging window therein which is configured to pass light from the light source of the microscope, through the sample (on slide 104), to the one or more imaging devices. The orifice may be provided in a central area, e.g., below the slide, when the adaptor is configured for placement on the microscope stage. In some embodiments, as shown in FIG. 18A, two sets of perpendicular slots may be formed in the body such that the body may be rotated relative to the microscope and still support the sample for imaging while magnetic actuation is performed. That is, a first set of slots may be positioned in a generally horizonal direction on either side of the adaptor body, and a second set of slots may be positioned in a generally vertical direction (in the illustrated case, through the horizontal slots, forming a cross—like shape). This allows the adaptor body 102A and the slide 104 to be positioned in two manners, i.e., a first position as shown in FIG. 18A, or a second position, whereby the adaptor body 102A is rotated about 90 degrees (i.e., towards the right in FIG. 18A) using the illustrated example.

Similarly, FIG. 18B shows another example of an adaptor with an adaptor body 102B including base 103 and sample holder 106B for horizontal application of a magnetic field. The adaptor body 102B similarly holds a conventional microscope slide 104 therein via sliding into the sample holder 106B, such that the sample is positioned to receive and pass light from a light source, through the slot(s) of the collar portion(s) 120, to the one or more imaging devices of the microscope, when the adaptor is mounted therein. In an embodiment, the sample holder 106B may be formed by corresponding slots 107 provided in opposite sides of the adaptor body. The adapter body 102B also includes an orifice 108 or imaging window therein which is configured to pass light from the light source of the microscope, through the sample (on slide 104), to the one or more imaging devices. As shown, the orifice may be provided in a central area, e.g., below the slide, when the adaptor is configured for placement on the microscope stage.

The system comprises an electromagnet integrated into one or more surfaces of the adaptor body, and/or coupled to the adaptor body in other ways. The electromagnet is configured to generate a magnetic field that interacts with the sample. The magnetic field is configured to be changed to actuate and/or modulate the field to change the sample while the sample is being imaged, allowing for control, i.e., dynamic actuation and tuning, of the magnetic field. When a magnetic field is applied, the magnetogel (hydrogel) microstructure will rearrange at the nanoscale, changing or modifying the (3D) culture matrix stiffness in situ. The upper limit on nanoparticle concentration may be determined by the uniformity of nanoparticle dispersion in the cured hydrogel matrix.

In some embodiments, the electromagnet is configured to generate a uniform magnetic field across the sample. In other embodiments, the magnetic field may be a time-varying magnetic field or magnetic field gradient applied to the sample. In some embodiments, the electromagnet comprises a Helmholtz coil system. In some embodiments, the sample comprises a microscope slide, a hydrogel, magnetic particles, and/or other components.

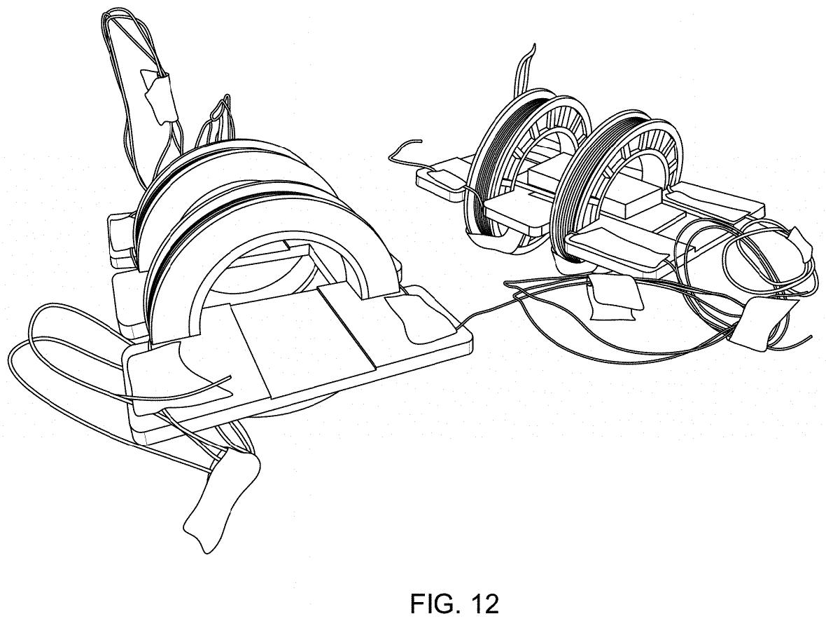

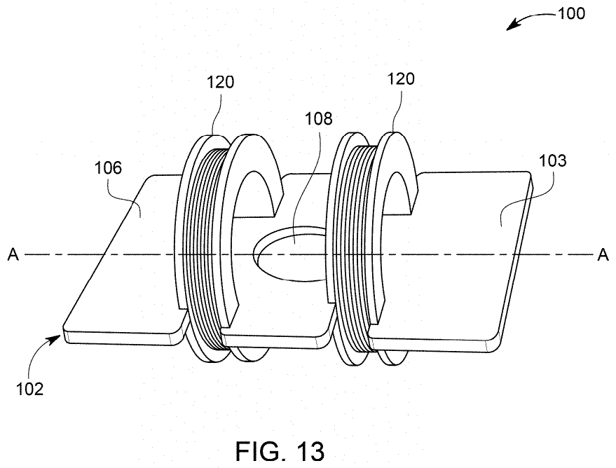

By way of a non-limiting example, a Helmholtz coil system may be adapted to fit on a microscope stage to allow for magnetic actuation while imaging. Example embodiments are shown in FIG. 12 and FIG. 18A, wherein the coil is optimized around ensuring a magnetic field across the entire hydrogel (on the slide 104 with the sample) while leaving space for optical (fluorescent) imaging. In this exemplary illustrated embodiment, a uniform magnetic field may be ensured and applied across the hydrogel for interaction with the sample via the structure of the adaptor and placement of the coil(s) therein. The design accommodates a microscope slide and ensures a transparent surface for imaging applications. As previously noted, the design of FIG. 18A also supports horizontal and vertical application of a magnetic field and can be configured in both directions. Accordingly, in addition to the y-direction application of a magnetic field (as presented in the embodiments of adaptor(s) shown in FIGS. 5-15, for example), FIG. 18A also supports z-direction application of the magnetic field.

By using 3D printing for initial test-fits of the design and then a higher resolution resin printer for the final structure, the design may be adapted for multiple microscopes and organoid sample holders. Finite difference time domain modeling of the electromagnetic field distribution across the sample may be performed for comparison.

As shown in the exemplary embodiment of FIG. 7, for example, the orifice 108 in the adaptor body 102 may be formed by, near, or adjacent to two or more corresponding collar portions 120 of the body, formed on opposite sides of the orifice 108 along an (imaginary) axis A-A in the longitudinal or length direction of the body 102. In an embodiment, the two collar portions 120 may be formed on opposite ends of an (imaginary) axis A-A that extends along a length of the body. The collar portions 120 are designed to extend radially in at least one direction relative to axis A-A, outwardly from base 103 of the adaptor body 102. Portions of the electromagnet (e.g., the Helmholtz coil) may be coupled to surfaces of these collars so that the electromagnet can produce the magnetic field described herein. In an embodiment, as shown in FIGS. 6-7 and 13-15, the collar portions 120 may include a generally rounded or circular surface that extends circumferentially around the base 103 of the adaptor body 102. This allows wire to be wound circularly and relatively around the base 103 of the adaptor body 102, such as represented in FIG. 12. The circular surface may be flanked by walls extending radially outward therefrom, so that the wire wound relatively around the circular surface is contained within the walls (see, e.g., FIG. 12). The circular surface of the collar portions 120 as depicted in the FIGS., however, is not intended to be limiting. In accordance with an embodiment, the collar portions 120 may include an ovular or rounded surface. In another embodiment, each the collar portions 120 may only include a semi-circular surface (or semi-round surface) that extends upwardly relative to the base 103.

Loops of wire are coiled around the collar portions 120 in order to form the coils of the electromagnet. The collar portions 120 are sized in order to fit a number N of loops thereon. In an embodiment, N may be between approximately 1 loop to approximately 3000 loops of wire on each collar portion 120. In one embodiment, N may be between approximately 200 loops to approximately 1000 loops of wire on each collar portion 120. In another embodiment, N=300 loops of wire on each collar portion 120.

The electric current may travel in a direction around the collar/orifice as shown by arrow EC in FIG. 18A, and/or in other directions, based on the winding of the loops. In the example of FIG. 18A, this creates a uniform magnetic field oriented in a vertical direction. These orientations and/or directions may be adjusted as necessary, depending on the application for which the present system is used. For example, in the positions of the adaptor body 102 as shown in FIGS. 7-15, the uniform magnetic field will be oriented in a horizontal direction.

Coupling of the portions of the electromagnet to the adaptor body may be facilitated by slots formed in the surfaces of the collars (e.g., so that wire may be coiled around the collar in a slot), adhesive, and/or by other methods.

In an embodiment, the walls and rounded or circular surface of the adaptor may be dimensioned to fit within the imaging area of a microscope. In one embodiment, a width WW of each of the collar portions 120 (taken between edges of the extending wall, as shown in FIG. 8) is approximately 65.0 mm +/−5.0 mm to approximately 80.0 mm +/−5.0 mm. In a particular embodiment, the width WW is approximately 70.0 mm. In an embodiment, the radius R of each collar portion is approximately 1.0 mm +/−0.5 mm to 400.0 mm +/−5.0 mm. In one embodiment, the radius R of each collar portion is approximately 30.0 mm +/−5.0 mm to 40.0 mm +/−5.0 mm. In a particular embodiment, the radius R is approximately 35.0 mm. In an embodiment, the radius of the coils/collar portions 120, and/or any other dimensions, may be based on the base sizing of the adaptor body. In an embodiment, the dimensions and/or radius of the coils/collar portions 120 may be configured or determined based on the type or size of sample, and/or configured or determined based on the type and/or dimensions of the microscope (and the microscope stage that the adaptor is removably attached to). In an embodiment, a distance DW (see FIG. 10) between the walls of each collar portion 120, which may also be referred to as a length of the circular surface thereof, is approximately 10.0 mm +/−5.0 mm to approximately 30.0 mm +/−5.0 mm. In an embodiment, a distance DC (see FIG. 10) between the collar portions 120 is approximately 20.0 mm +/−5.0 mm to approximately 40.0 mm +/−5.0 mm. In a particular embodiment, the distance DC is approximately 30.0 mm. A depth DA (or thickness) of the adaptor, i.e., a height of the slot/area formed in the Z-direction from the circular surface to the top of the walls that flank it (see FIG. 10), may vary from approximately 1 mm to approximately 30 mm, for example.

In one embodiment, the collar portions 120 for the coil have dimensions (length and depth) of 10 mm×10 mm with a radius of 35 mm. In another embodiment, the coil dimensions (length and depth) may be 30 mm×30 mm. In still yet another embodiment, the coil dimensions (length and depth) may be 1 mm×1 mm. Again, such dimensions are not intended to be limiting and may be configured or determined based on the type or size of sample, and/or configured or determined based on the type and/or dimensions of the microscope (and the microscope stage that the adaptor is removably attached to).

Coils include the physical mount supports as well as the number of loops of wiring on the mount. Using the coil dimensions and number of loops in the prototype, finite element method (FEM) modeling of the electromagnetic field distribution has been performed and a comparison between the modeling to experimental measurements has been made. Notably, the initial results (not oscillating the field strength) are uniform across the sample location when the electromagnets are operated in the same direction even after running the system for 3 hours.

The Helmholtz coil system presented in the Figures may be optimized for tissue samples to allow for magnetic actuation while imaging. To allow dynamic switching of the field, the electromagnet is controlled with a power source connected to a function generator (signal generator). In one embodiment, the loops (wires) of the electromagnet may be connected to two power sources (one per coil) via alligator clips or other connecting devices. In an embodiment, there may be direct interface between the loops and a respective power supply (i.e., no connecting device). The number of power sources may be varied and is not limiting.

In an embodiment, a mechano-electrical or an electrical control system may be associated with the adapter and system. Such a control system may, for example, be used for feedback or feedforward loop for current control. Further, it is envisioned in embodiments that other control parameters and devices may be utilized, which may include, but are not limited to: components that turn on/off (actuate) the current, components that modulate the current without using the knobs on the associated power source, on-device power to supply to the wire loops, remote control of supplied current to create the magnetic field, and/or a control device that alters and/or switched frequency of supplied current or applied magnetic field. In one embodiment, control may be initiated via a computing system, such as system 600 noted later below.

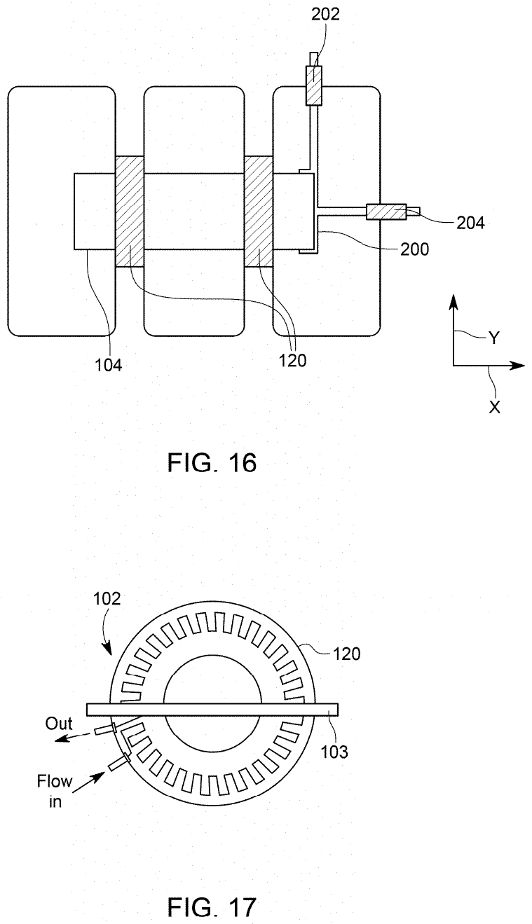

While it may be understood that, in some embodiments, the microscope slide 104 may be manually moved within the adaptor, in some embodiments, the system may include a device provided for movement of a slide within the adaptor, e.g., in order to limit contact and/or contamination of the sample and hydrogel. FIG. 16 illustrates a schematic of an exemplary design for physically moving a slide with a sample within an adaptor, in accordance with an embodiment. Such features may be utilized with any of the herein disclosed adaptor designs. FIG. 16 shows an example of a clamp 200 that includes a frame and a jaw for capturing a portion (in this illustration, an end or side; on the right) of a slide 104. Such a clamp 200 may include a fixed jaw and a movable jaw, for example, much like a standard clamp, or may be a vacuum clamp, or any other type of device configured to hold and correspondingly move the microscope slide 104 for positioning on the microscope stage. The clamp 200 may be configured for movement, e.g., via one or more motors, screws, etc., attached thereto. Movement of the clamp may be performed via manual and/or electric/electro-mechanical devices and is not limited. For example, in an embodiment, a y-direction motor 202 may be provided and connected to the clamp 200 to move the slide 104 in the Y-direction, or along a width, and an x-direction motor 204 may be provided and connected to the clamp 200 to move the slide in the X-direction, or along a length/longitudinal axis A-A. In accordance with an embodiment, the motors may be electronically controlled.

It should be noted that the material used to form the adaptor may assist in providing cooling properties during operation of the electromagnet. For example, forming the adaptor using a high temperature 3D printer resin assists in accounting for temperature changes during application of current to the wires. Further, in accordance with embodiments herein, one or more cooling mechanisms may be associated with the system for cooling purposes, in addition or as an alternative to, the type of material used to form the adaptor. In an embodiment, forms of air flow cooling are used in the disclosed system. For example, fan(s) may be installed on the adaptor and/or around the adaptor (e.g., around a mounting area, on/around the microscope stage) of to decrease temperature of the wires, in accordance with an embodiment. In an embodiment, gaps may be utilized in the coil material support to increase area of wire loops exposed to ambient air to enable convective heat transfer. For example, as seen in the illustrative embodiments of FIGS. 7-8 and 11, the walls of the collar portions 120 may include gaps or openings therein (see, e.g., generally rectangular cut-out portions) to promote air flow around the coils. Such gaps may be used alone or in combination with fan(s), for example.

In another embodiment, cooling properties may be utilized via a fluid coolant or a liquid coolant flow path provided in the adaptor. For example, coolant lines may be added to the adaptor body 102 in a number of areas, e.g., lines looped around the coils, wire loops, and/or base of the mount, to promote cooling. FIG. 17 illustrates just one example of an inlet, an outlet, and coolant lines for placement within the adaptor. A pump (not shown) may be provided to distribute a flow of coolant through the adaptor. In one embodiment, the coolant lines may be provided in the form of added microfluidic and/or millifluidic channels within the adaptor to allow flow of coolant material therethrough. Such channels may be provided in the circular/rounded surfaces and/or walls of the collar portions 120, in accordance with an embodiment. The fluid or liquid used for cooling is not limited. In an embodiment, the coolant may be water.

Moreover, physical cooling structures may be used in the disclosed system to assist in cooling, in accordance with embodiments herein. For example, one or more of the following may be provided for added cooling features: addition of fins to the adaptor (via coils or base of the adaptor), addition of any heat sink to the adaptor, and/or addition of a cold plate (in some instances, in addition to use of a fluid/liquid coolant). In embodiments, thermoelectric cooling features may be provided as part of the disclosed system, which may include, for example, addition of Peltier device for cooling to the coils or base of the adaptor and/or addition of an electrostatic fluid accelerator to the coils or base of the adaptor.

As such, it should be understood that the above examples for providing cooling features and properties to the disclosed system are not limiting.

With the disclosed adaptor and system, movement of individual cells may be tracked and analyzed.

To verify the results of the disclosed system, several forms of testing have been implemented:

Benchmark (static) system testing—the magnetic hydrogel system may be characterized according to typical strain deformations of the hydrogel system. The deformation of organoids influenced by the physical deformation force applied by a standard load-frame may be characterized. Fixed strain points as defined by previous mechanical testing and characterization of the hydrogel matrix may be used. This in situ DAPI for nucleus and GFP for cytoskeleton facilitates characterization of the typical behavior of organoids when exposed to deformation forces.

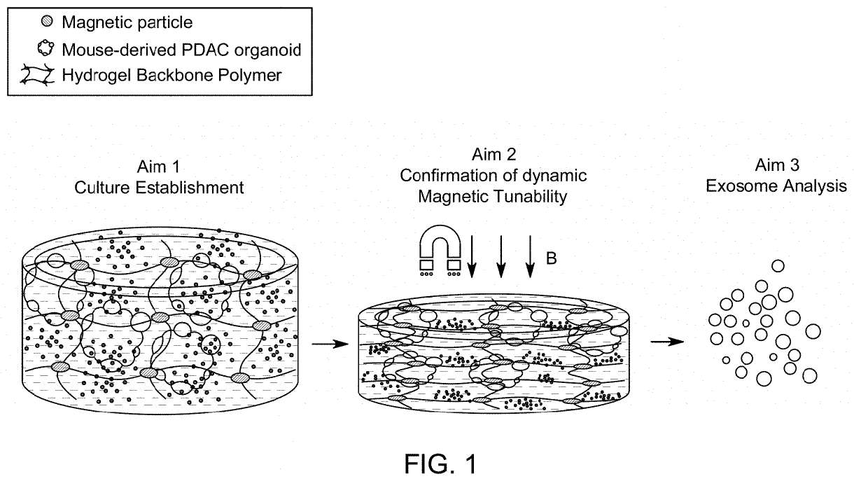

Dynamic testing (confirmation) of the magnetically tunable hydrogel system—benchmark testing may be repeated, using the electromagnet system to apply the magnetic field. FIG. 6 presents a conceptual image of what an embodiment of the present system may look like when incorporated on a microscope stage, showing polymers and nanoparticles that are developed and optimized to establish cultures in a hydrogel like that of FIG. 1.

With organoids fluorescently stained, the (present) electromagnet system may be removably attached to the microscope stage and may image the organoids within the hydrogel with stepwise increases in the current supplied across the wire coils to a predetermined maximum magnetic field strength. Each step may allow for enough time to capture z-stack information of the organoids.

Using the data gathered as described herein, the system may perform image analysis on the z-stacks/videos acquired of the system to compare the relative forces and behavior of the hydrogel with dynamic actuation. The efficacy of the magnetic field actuation may be comparable to the physical strain applied as described above, for example.

Quantify the Changes in Secreted Exosomes Derived from PDAC Organoids Cultured in the Magnetically Tunable Hydrogel Matrix

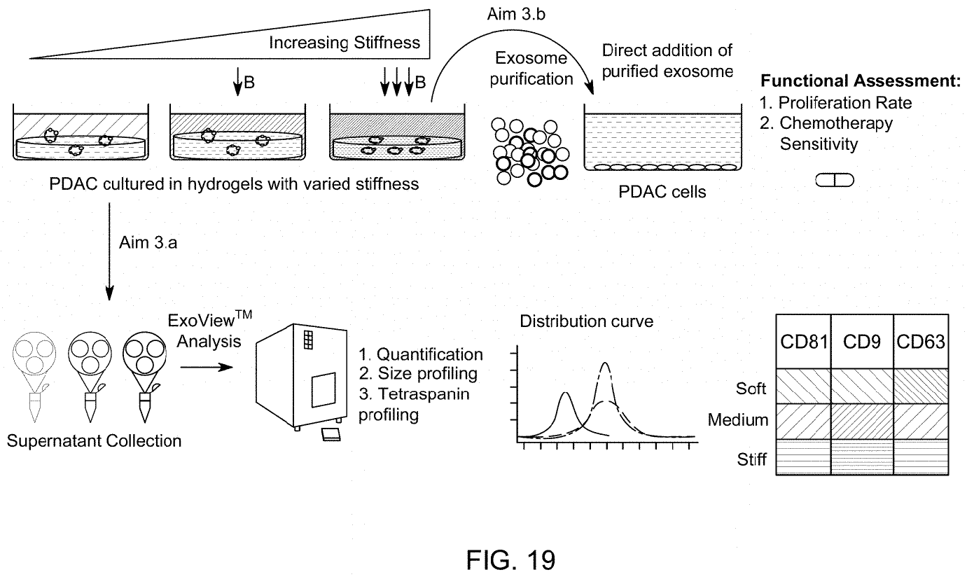

Previous research has focused on how exosomes affect tumor growth and chemoresistance, but they mainly ignored whether variation in tissue stiffness would affect exosome secretion quantitatively and qualitatively. Given the fact that around 3-fold tissue stiffening was observed in PDAC bulk tumor compared to normal pancreas tissue, the structurally altered surrounding matrix changes the secretion rate, profile, and function of PDAC-derived exosomes, as illustrated in FIG. 19.

Qualitative Analysis of Exosome Secretion of PDAC Organoids Cultured in Different Stiffnesses

Quantitatively measuring the exosome secretion may be performed by culturing mouse derived PDAC organoids in the proposed magnetically tunable hydrogel matrix while attenuating the magnetic field to induce microstructure matrix changes surrounding the organoids. Supernatant may be collected and loaded to ExoView™ to analyze exosome quantity, size distribution and tetraspanin (CD9, CD63, CD81, etc.) profile.

Functional analysis of isolated exosome from PDAC cultured in different stiffnesses

To functionally investigate the effect of PDAC-organoid derived exosomes under the influence of magnetic field actuation (M-Exos) on recipient cells, M-Exos may be collected and add them to PDAC cells in culture. Growth parameters including absolute viable cell number may be quantified over time through a live cell imaging system. Cultures may also be treated with commonly used chemotherapy agents like GEM to test how addition of M-Exos from different stiffness conditions affects chemoresistance. At the end of the drug test, cell lysates may be collected to verify chemoresistance through cleaved polyADP polymerase (cl-PARP), phosphorylation of AKT and MAPK through Western blot, and these methods are established as described previously.

From a clinical translation perspective, the applicability of the proposed approach is linked to a critical need to understand the biomechanical response of cancer cells to their environment and the drugs used in attempts to eradicate these cells. This is a field that researchers have been trying to elucidate, and the present system could be a catalyst for numerous investigators. While an initial target is PDAC, this system could be used to explore nearly any type of cancer to monitor tumor response to a wide range of therapeutics via exosome secretion. This system could be particularly powerful when paired with patient-derived xenograft (PDX) models and organoids.

Analysis of Tissues

As another example, the system has applicability to numerous programs including, but not limited to, performing measurements using 2D bacterial populations and 3D neural microtissues. These measurements set the stage for future work in Microbiology and in Neuroscience, including:

Microbiology: The mechanical environment surrounding bacteria is known to impact motility and gene expression, but open questions remain as to exactly how bacterial cells sense and respond to surface mechanical cues. A lack of experimental tools has prevented studies of the mechanosensitive responses and migratory strategies for complex surfaces, including surfaces with dynamic mechanical properties. Such studies would reveal how information about the mechanical microenvironment is integrated to influence cell-behavior as cells navigate a spatiotemporal mechanical landscape. Accordingly, the disclosed system provides tools for such studies.

Neuroscience: While it is known that neural cells have mechanoreceptors, the relationship between mechanical forces and neural activity is not completely understood. One of the most commonly used methods to analyze neural activity is optical imaging with indicator dyes. Therefore, the development of a method that would be directly compatible with optical imaging has high potential to impact studies of neural development, maturation, injury and disease. Here, the method implemented by using the disclosed system may indeed enable greater understanding of such relationships.

Recent work has revealed that mechanical properties of the microenvironment strongly influence cell growth, biofilm formation, and cell migration. Current experimental methods to probe these mechanosensory effects include growing cells on surfaces with defined and static mechanical properties, examination of cells under fluid flow, and encapsulation of cells within gel matrices. In real-world contexts, cells experience spatial and temporal changes in the mechanical microenvironment. Understanding the response to such complex mechanical environments using the disclosed system may reveal how cells regulate broad phenotypic responses, such as biofilm formation and migration, within realistic contexts.

Notably, this system offers new capabilities that have transformative potential for the biomedical research community. In particular, the disclosed dynamically tunable hydrogel matrix coupled with the electromagnetic microscope adaptor will allow the time-dependent nature of mechanobiology effects to be studied in multi-cellular samples. Measurements may be performed by comparing across samples with different preparations or by relying on non-reversible mechanical changes.

Accordingly, this disclosure describes a system that generates magnetic field configured to removably couple with a microscope for magnetically actuating a sample while imaging the sample with the microscope. As described herein, in accordance with embodiments, the sample includes a hydrogel base and magnetic nanoparticles held by the hydrogel base. The system includes an adaptor body configured to removably couple with a microscope stage, the adaptor body having an orifice configured to pass light from a light source of the microscope, through the sample, to one or more imaging devices of the microscope; a sample holder formed by the adaptor body, configured to hold the sample such that the sample is positioned to receive and pass the light from the light source to the one or more imaging devices; and an electromagnet integrated into one or more surfaces of the adaptor body, the electromagnet configured to generate a magnetic field that interacts with the sample, the magnetic field being configured to be changed and/or modulated to actuate and/or change the sample while the sample is being imaged.

Further, it should be understood that this disclosure also provides a method for facilitating magnetic actuation of a sample while imaging the sample with a microscope. A microscope slide is inserted into the adaptor body and aligned (and/or secured) therein, relative to the orifice, for imaging, either before or after placement of the adaptor and its body into the microscope. Accordingly, the method for facilitating magnetic actuation of a sample on the microscope slide may include, for example (in no specific order): coupling the adaptor body to a microscope stage, the adaptor body configured to removably couple with the microscope stage; forming a sample holder with the adaptor body configured to hold the sample such that the sample is positioned to receive and pass the light from the light source to the one or more imaging devices; forming the sample by generating a magnetically responsive hydrogel extracellular matrix model; and providing an electromagnet integrated into one or more surfaces of the adaptor body, the electromagnet configured to generate a magnetic field that interacts with the sample, the magnetic field configured to be changed and/or modulated to actuate and/or change the sample while the sample is being imaged.

In some embodiments, the present system(s) may be or include computing system, machine learning technology, a neural network or other model that is trained and configured to predict information, and or other electronic resources. As an example, neural networks may be based on a large collection of neural units (or artificial neurons). Neural networks may loosely mimic the manner in which a biological brain works (e.g., via large clusters of biological neurons connected by axons). Each neural unit of a neural network may be simulated as being connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

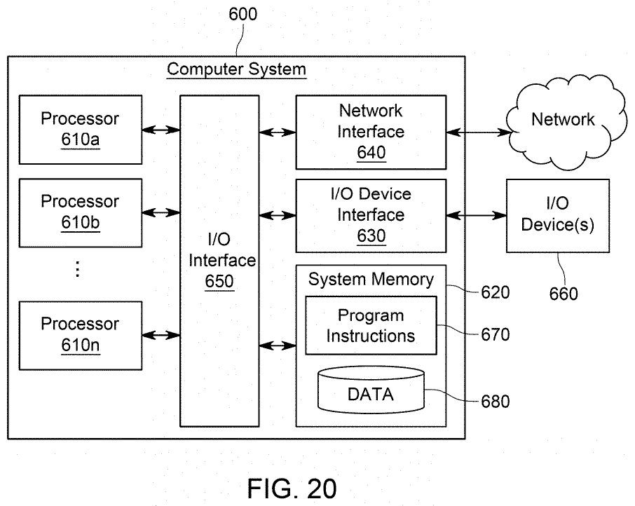

In some embodiments, the present system(s) and method(s), and/or portions of the present system(s) and method(s) may be executed in a single computing device, or in a plurality of computing devices in a datacenter, e.g., in a service oriented or micro-services architecture. FIG. 20 is a diagram that illustrates an exemplary computing system 600 in accordance with embodiments of the present system. Various portions of systems and methods described herein, may include or be executed on one or more computer systems the same as or similar to computing system 600. For example, the present system itself, a mobile user device, a desktop user device, external resources, and/or other components of the system may be and/or include one more computer systems the same as or similar to computing system 600. Further, processes, modules, processor components, and/or other components of the system described herein may be executed by one or more processing systems similar to and/or the same as that of computing system 600.

Computing system 600 may include one or more processors (e.g., processors 610a-610n) coupled to system memory 620, an input/output I/O device interface 630, and a network interface 640 via an input/output (I/O) interface 650. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 600. A processor may execute code (e.g., processor firmware, a protocol stack, a database management system, an operating system, or a combination thereof) that creates an execution environment for program instructions. A processor may include a programmable processor. A processor may include general or special purpose microprocessors. A processor may receive instructions and data from a memory (e.g., system memory 620). Computing system 600 may be a uni-processor system including one processor (e.g., processor 610a), or a multi-processor system including any number of suitable processors (e.g., 610a-610n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field programmable gate array) or an ASIC (application specific integrated circuit). Computing system 600 may include a plurality of computing devices (e.g., distributed computer systems) to implement various processing functions.

I/O device interface 630 may provide an interface for connection of one or more I/O devices 660 to computer system 600. I/O devices may include devices that receive input (e.g., from a user) or output information (e.g., to a user). I/O devices 660 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. I/O devices 660 may be connected to computer system 600 through a wired or wireless connection. I/O devices 660 may be connected to computer system 600 from a remote location. I/O devices 660 located on remote computer system, for example, may be connected to computer system 600 via a network and network interface 640.

Network interface 640 may include a network adaptor that provides for connection of computer system 600 to a network. Network interface may 640 may facilitate data exchange between computer system 600 and other devices connected to the network. Network interface 640 may support wired or wireless communication. The network may include an electronic communication network, such as the Internet, a local area network (LAN), a wide area network (WAN), a cellular communications network, or the like.

System memory 620 may be configured to store program instructions 670 or data 680. Program instructions 670 may be executable by a processor (e.g., one or more of processors 10a-610n) to implement one or more embodiments of the present techniques. Instructions 670 may include modules and/or components of computer program instructions for implementing one or more techniques described herein with regard to various processing modules and/or components. Program instructions may include a computer program (which in certain forms is known as a program, software, software application, script, or code). A computer program may be written in a programming language, including compiled or interpreted languages, or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 620 may include a tangible program carrier having program instructions stored thereon. A tangible program carrier may include a non-transitory computer readable storage medium. A non-transitory computer readable storage medium may include a machine readable storage device, a machine readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random access memory (SRAM), synchronous dynamic RAM (SDRAM)), bulk storage memory (e.g., CD-ROM and/or DVD-ROM, hard-drives), or the like. System memory 620 may include a non-transitory computer readable storage medium that may have program instructions stored thereon that are executable by a computer processor (e.g., one or more of processors 610a-610n) to cause the subject matter and the functional operations described herein. A memory (e.g., system memory 620) may include a single memory device and/or a plurality of memory devices (e.g., distributed memory devices). Instructions or other program code to provide the functionality described herein may be stored on a tangible, non-transitory computer readable media. In some cases, the entire set of instructions may be stored concurrently on the media, or in some cases, different parts of the instructions may be stored on the same media at different times, e.g., a copy may be created by writing program code to a first-in-first-out buffer in a network interface, where some of the instructions are pushed out of the buffer before other portions of the instructions are written to the buffer, with all of the instructions residing in memory on the buffer, just not all at the same time.

I/O interface 650 may be configured to coordinate I/O traffic between processors 610a-610n, system memory 620, network interface 640, I/O devices 660, and/or other peripheral devices. I/O interface 650 may perform protocol, timing, or other data transformations to convert data signals from one component (e.g., system memory 620) into a format suitable for use by another component (e.g., processors 610a-610n). I/O interface 650 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 600 or multiple computer systems 600 configured to host different portions or instances of embodiments. Multiple computer systems 600 may provide for parallel or sequential processing/execution of one or more portions of the techniques described herein.

Those skilled in the art will appreciate that computer system 600 is merely illustrative and is not intended to limit the scope of the techniques described herein. Computer system 600 may include any combination of devices or software that may perform or otherwise provide for the performance of the techniques described herein. For example, computer system 600 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, a television or device connected to a television (e.g., Apple TV™), or a Global Positioning System (GPS), or the like. Computer system 600 may also be connected to other devices that are not illustrated, or may operate as a stand-alone system. In addition, the functionality provided by the illustrated components may in some embodiments be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory and other storage devices for purposes of memory management and data integrity. Alternatively, in other embodiments some or all of the software components may execute in memory on another device and communicate with the illustrated computer system via inter-computer communication. Some or all of the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 600 may be transmitted to computer system 600 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present invention may be practiced with other computer system configurations.

Components of the present system may be described as discrete functional blocks, but embodiments are not limited to systems in which the functionality described herein is organized as described. The functionality provided by each of the components may be provided by software or hardware modules that are differently organized than is presently described, for example such software or hardware may be intermingled, conjoined, replicated, broken up, distributed (e.g. within a data center or geographically), or otherwise differently organized. The functionality described herein may be provided by one or more processors of one or more computers executing code stored on a tangible, non-transitory, machine readable medium. In some cases, notwithstanding use of the singular term “medium,” the instructions may be distributed on different storage devices associated with different computing devices, for instance, with each computing device having a different subset of the instructions, an implementation consistent with usage of the singular term “medium” herein. In some cases, third party content delivery networks may host some or all of the information conveyed over networks, in which case, to the extent information (e.g., content) is said to be supplied or otherwise provided, the information may be provided by sending instructions to retrieve that information from a content delivery network.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

The phrase “induced by”, encompasses “worsened by”, “aggravated by”, “exacerbated by”, and/or “magnified by”, unless clearly indicated otherwise.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in some embodiments or aspects of the methods disclosed herein, some materials and/or method steps are excluded. Thus, even though the invention is generally not expressed herein in terms of what the invention does not include aspects that are not expressly excluded in the invention are nevertheless disclosed herein.

Some embodiments of the technology described herein suitably can be practiced in the absence of an element not specifically disclosed herein. Accordingly, in some embodiments the term “comprising” or “comprises” can be replaced with “consisting essentially of” or “consisting of” or grammatical variations thereof. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”,“at least 97%”,“at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

We claim:

1. A system configured to facilitate magnetically actuating a sample, while imaging the sample with a microscope, the system comprising:
an adaptor body configured to removably couple with a microscope stage, the adaptor body comprising an orifice configured to pass light from a light source of the microscope, through the sample, to one or more imaging devices of the microscope;
a sample holder formed by the adaptor body and configured to hold the sample such that the sample is positioned to receive and pass the light from the light source to the one or more imaging devices; and
an electromagnet integrated into one or more surfaces of the adaptor body, the electromagnet configured to generate a magnetic field that interacts with the sample, the magnetic field being configured to be changed and/or modulated to actuate and/or change the sample while the sample is being imaged.

2. The system of claim 1, wherein the electromagnet is configured to generate a uniform magnetic field across the sample.

3. The system of claim 1, wherein the electromagnet comprises a Helmholtz coil system.

4. The system of claim 1, wherein the sample comprises a glass slide or a plastic slide.

5. The system of claim 1, wherein the sample comprises a magnetically responsive hydrogel extracellular matrix model.

6. The system of claim 5, wherein the sample comprises magnetic nanoparticles and a hydrogel base.

7. The system of claim 6, wherein the magnetic nanoparticles comprise manganese-doped iron oxide nanoparticles or cobalt-doped iron oxide nanoparticles.

8. A sample configured to be magnetically actuated while being imaged with a microscope, the sample comprising:
a hydrogel base; and
magnetic nanoparticles held by the hydrogel base, the magnetic nanoparticles configured to be actuated by a magnetic field that interacts with the sample, the magnetic field configured to be changed and/or modulated to actuate and/or change the sample while the sample is being imaged.

9. The sample of claim 8 in combination with a system, wherein the magnetic field is generated by the system configured to removably couple with the microscope, the system comprising:
an adaptor body configured to removably couple with a microscope stage, the adaptor body comprising an orifice configured to pass light from a light source of the microscope, through the sample, to one or more imaging devices of the microscope;
a sample holder formed by the adaptor body and configured to hold the sample such that the sample is positioned to receive and pass the light from the light source to the one or more imaging devices; and
an electromagnet integrated into one or more surfaces of the adaptor body, the electromagnet configured to generate the magnetic field.

10. The sample of claim 9, wherein the electromagnet of the system is configured to generate a uniform magnetic field across the sample.

11. The sample of claim 9, wherein the electromagnet of the system comprises a Helmholtz coil system.

12. The sample of claim 8, wherein the sample further comprises a glass slide or a plastic slide.

13. The sample of claim 8, wherein the sample comprises a magnetically responsive hydrogel extracellular matrix model.

14. The sample of claim 8, wherein the magnetic nanoparticles comprise manganese-doped iron oxide nanoparticles or cobalt-doped iron oxide nanoparticles.

15. A method for facilitating magnetic actuation of a sample while imaging the sample with a microscope, the method comprising:
coupling an adaptor body to a microscope stage, the adaptor body configured to removably couple with the microscope stage, the adaptor body comprising an orifice configured to pass light from a light source of the microscope, through the sample, to one or more imaging devices of the microscope;
forming a sample holder with the adaptor body configured to hold the sample such that the sample is positioned to receive and pass the light from the light source to the one or more imaging devices;
forming the sample by generating a magnetically responsive hydrogel extracellular matrix model; and
providing an electromagnet integrated into one or more surfaces of the adaptor body, the electromagnet configured to generate a magnetic field that interacts with the sample, the magnetic field configured to be changed and/or modulated to actuate and/or change the sample while the sample is being imaged.

16. The method of claim 15, wherein the electromagnet is configured to generate a uniform magnetic field across the sample.

17. The method of claim 15, wherein the electromagnet comprises a Helmholtz coil system.

18. The method of claim 15, wherein the sample comprises magnetic nanoparticles and a hydrogel base.

19. The method of claim 18, wherein the magnetic nanoparticles comprise manganese-doped iron oxide nanoparticles or cobalt-doped iron oxide nanoparticles.
